(19) United States
(12) Patent Application Publication
(10) Pub. No.: US 2009/0054941 A1
(43) Pub. Date: Feb. 26, 2009

(54) STIMULATION FIELD MANAGEMENT

(75) Inventors: Michael D. Eggen, Lake Elmo, MN (US); John L. Sommer, Coon Rapids, MN (US); Michael Ebert, Fridley, MN (US); David Wayne Bourn, Maple Grove, MN (US); Gabriela C. Miyazawa, Fridley, MN (US)

Correspondence Address: Medtronic, Inc., 710 Medtronic Parkway, Minneapolis, MN, 55432, US

(73) Assignee: Medtronic, Inc.

(21) Appl. No.: 12/195,313

(22) Filed: Aug. 20, 2008

Related U.S. Application Data
(60) Provisional application No. 60/956,832, filed on Aug. 20, 2007.
(60) Provisional application No. 60/956,868, filed on Aug. 20, 2007.
(60) Provisional application No. 61/049,240, filed on Apr. 30, 2008.

(51) Int. Cl. A61N 1/05; A61N 1/362

(52) U.S. Cl. 607/9; 607/116; 607/2

(57) ABSTRACT

This disclosure describes techniques for controlling a depth of propagation of a stimulation field extending from an outer diameter of a lead body of an implantable stimulation lead. An implantable electrical stimulation lead may include a lead body, and at least one electrode arranged as a ring. An outer diameter of the ring may be different than an outer diameter of the lead body. A ring with a diameter smaller than the diameter of the lead body may be useful in limiting the depth of propagation of the stimulation field within patient tissue. A ring with a diameter greater than the diameter of the lead body may be useful in extending the depth of propagation of the stimulation field.

This application claims the benefit of U.S. Provisional Application No. 60/956,832, filed Aug. 20, 2007, U.S. Provisional Application No. 60/956,868, filed Aug. 20, 2007 and U.S. Provisional Application No. 61/049,240, filed Apr. 30, 2008, each of which are hereby incorporated by reference.

### TECHNICAL FIELD

The present disclosure relates to medical devices, more particularly to implantable medical leads.

### BACKGROUND

In the medical field, implantable leads are used with a wide variety of medical devices. For example, implantable leads are commonly used to form part of implantable cardiac pacemakers that provide therapeutic stimulation to the heart by delivering pacing, cardioversion or defibrillation pulses. The pulses can be delivered to the heart via electrodes disposed on the leads, e.g., typically near distal ends of the leads. Leads may be configured to allow electrodes to be positioned at desired cardiac locations so that the pacemaker can deliver pulses to the appropriate locations. Leads are also used for sensing purposes, or for both sensing and stimulation purposes. Implantable leads are also used in neurological devices, muscular stimulation therapy, gastric system stimulators, and devices that sense chemical conditions in a patient's blood.

Pacing leads, such as left ventricle (LV) pacing leads, are typically placed in the coronary veins near the phrenic nerve. Phrenic nerve stimulation is generally undesirable during LV pacing therapy. In some instances, implantable leads may need to be specifically positioned to avoid phrenic nerve stimulation when using LV pacing therapy, which may result in a non-optimal LV pacing site for an implanted lead.

### SUMMARY OF THE DISCLOSURE

In general, the present disclosure is directed toward controlling a depth of propagation of a stimulation field extending from an outer diameter of a lead body of an implantable stimulation lead. An implantable stimulation lead according to an embodiment of the present disclosure includes one or more electrodes arranged as a ring. The ring has a different diameter than the lead body. A ring with a diameter smaller than the diameter of the lead body may be useful in limiting the depth of propagation of the stimulation field. A ring with a diameter greater than the diameter of the lead body may be useful in extending the depth of propagation of the stimulation field. The lead may be coupled to a cardiac stimulator or other medical device to deliver stimulation therapy to a patient. Controlling the depth of propagation of the stimulation field may be useful, for example, to avoid phrenic nerve stimulation during left ventricular (LV) pacing.

In one embodiment, an implantable electrical stimulation lead comprises a lead body and at least one electrode arranged as a ring, wherein an outer diameter of the ring being recessed from an outer diameter of the lead body.

In an embodiment, an implantable electrical stimulation lead comprises a lead body and at least one electrode arranged as a ring, wherein an outer diameter of the ring being greater than the lead diameter such that the ring protrudes relative to the lead body.

In another embodiment, a system comprises a cardiac medical device that delivers electrical stimulation and an implantable electrical stimulation lead, wherein the lead comprises a lead body and at least one electrode arranged as a ring, wherein an outer diameter of the ring differs from an outer diameter of the lead body.

In an embodiment, a method comprises implanting an electrical stimulation lead within a patient. The lead comprises a lead body, and at least one electrode arranged as a ring, wherein an outer diameter of the ring being recessed from an outer diameter of the lead body. The method further comprises delivering stimulation therapy to a tissue within the patient using the at least one electrode.

In yet another embodiment, an implantable electrical stimulation lead comprises a lead body and means for providing a stimulation field having a limited depth extending from an outer diameter of the lead body.

The details of one or more embodiments are set forth in the accompanying drawings and the description below. Other features, objects, and benefits of the present disclosure will be apparent from the description and drawings, and from the claims.

### BRIEF DESCRIPTION OF DRAWINGS

FIG. 1 is a conceptual diagram illustrating an example implantable medical device system.

FIG. 2 is a side view of an embodiment of a distal end of an implantable electrical stimulation lead including a recessed electrode.

FIG. 3 is a side view of an embodiment of a distal end of an implantable electrical stimulation lead including a recessed electrode and a shield over the recessed electrode.

FIG. 4A is a side view of an embodiment of a distal end of an implantable electrical stimulation lead including electrode segments that form a recessed ring and electrodes segments at the distal tip of the lead body.

FIG. 4B is a cross-sectional view of the lead of FIG. 4A illustrating the electrode segments that form the recessed ring.

FIG. 4C is another cross-sectional view of the lead of FIG. 4A illustrating the electrode segments at the distal tip of the lead body.

FIG. 5 is a side view of an embodiment of a distal end of an implantable electrical stimulation lead including an electrode that protrudes relative to the lead body.

FIG. 6 is a side view of an embodiment of a distal end of an implantable electrical stimulation lead illustrating electrical conductors within the lead body.

### DETAILED DESCRIPTION

The present disclosure is generally directed toward controlling a depth of propagation of a stimulation field extending from an outer diameter of a lead body of an implantable stimulation lead. An implantable stimulation lead according to an embodiment includes one or more electrodes arranged as a ring. The ring has a different diameter than the lead body. A ring with a diameter smaller than the diameter of the lead body may be useful in limiting the depth of propagation of the stimulation field. A ring with a diameter greater than the diameter of the lead body may be useful in extending the depth of propagation of the stimulation field. The lead may be coupled to a cardiac stimulator or other medical device to deliver stimulation therapy to a patient. Controlling the depth of propagation of the stimulation field may be useful, for example, to avoid phrenic nerve stimulation during left ventricular (LV) pacing.

While the description primarily refers to implantable electrical stimulation leads and implantable medical devices that deliver stimulation therapy to a patient's heart, e.g., pacemakers, defibrillators, and cardiac leads, the features of the leads described herein are useful with other types of medical devices and implantable electrical stimulation leads. For example, an implantable electrical stimulation lead according to an embodiment of the disclosure may take the form of an intravenous lead, intramuscular lead, subcutaneous lead, gastro-intestinal lead, pelvic lead, deep brain stimulation lead, cortical stimulation lead, spinal cord stimulation lead, or other neurostimulation leads. The leads described herein may be used with any medical device that delivers neurostimulation therapy (e.g., spinal cord stimulation or deep brain stimulation), stimulation to one or more muscles, muscle groups or organs, and, in general, stimulation to any tissue of a patient. In other applications, the leads described herein can be used for recording or monitoring, gene therapy, or other applications.

In addition, while the examples shown in the figures include leads coupled at their proximal ends to a stimulation therapy controller, e.g., implantable medical device, located remotely from the electrodes, other configurations are also possible and contemplated. In some examples, a lead comprises a portion of a housing, or a member coupled to a housing, of stimulation generator located proximate to or at the stimulation site, e.g., a microstimulator. In other examples, a lead comprises a member at stimulation site that is wirelessly coupled to an implanted or external stimulation controller or generator. For this reason, as referred to herein, the term of a “lead” includes any structure having one or more stimulation electrodes disposed on its surface.

Leads according to the present disclosure are not limited for use with pacemakers, cardioverters or defibrillators. For example, in other embodiments, the described techniques may be used with patient monitoring devices or devices that integrate monitoring and stimulation features. In those cases, leads may include different configurations, such as sensors disposed on distal ends of the respective lead for sensing patient conditions or other configurations of electrodes, depending on the type of target stimulation site or type of electrical stimulation therapy.

For example, for effective cardiac pacing, stimulation therapy must be of adequate energy for a given location to cause depolarization of the myocardium. Sensing a physiological parameter of the patient may be used to verify that pacing therapy has captured the heart, i.e., initiated a desired response to the therapy such as, for example, providing pacing, resynchronization, defibrillation and/or cardioversion. Such sensing may include sensing an evoked R-wave or P-wave after delivery of pacing therapy, sensing for the absence of an intrinsic R-wave or P-wave prior to delivering pacing therapy, or detecting a conducted depolarization in an adjacent heart chamber.

These and other physiological parameters may be sensed using electrodes that may be also used to deliver stimulation therapy. For example, a system may sense physiological parameters using the same electrodes used for providing stimulation therapy or electrodes that are not used for stimulation therapy. As with stimulation therapy, selecting which electrode(s) are used for sensing physiological parameters of a patient may alter the signal quality of the sensing techniques. For this reason, sensing techniques may include one or more algorithms to determine the suitability of each electrode or electrode combination in the stimulation therapy system for sensing one or more physiological parameters. Sensing physiological parameters may also be accomplished using electrode or sensors that are separate from the stimulation electrodes, e.g., electrodes capable of delivering stimulation therapy, but not selected to deliver the stimulation therapy that is actually being delivered to the patient.

Accordingly, one or more electrodes arranged as a ring that has a different diameter than the lead body may be selected used, for example, for delivery of electrical stimulation, sensing electrical signals, such as an electrocardiogram for the reasons mentioned above, impedance measurements, or uses known for implanted electrodes in the art. Electrodes so arranged may provide benefits with respect to stimulation delivery, as discussed herein, and may also provide benefits when used for such other purposes.

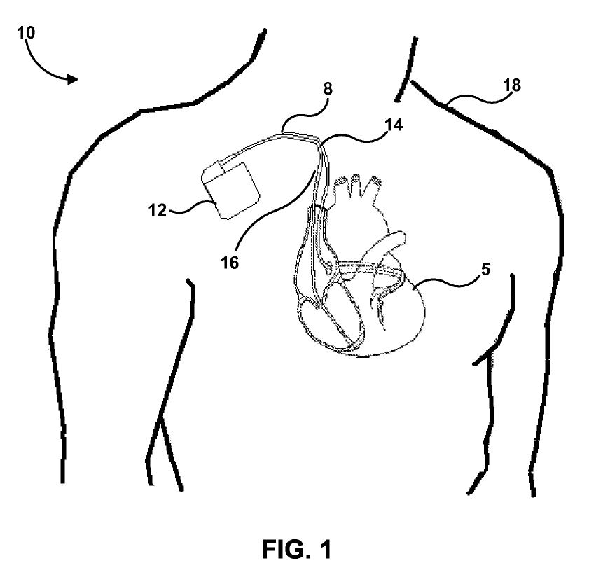

FIG. 1 is a conceptual diagram illustrating an example implantable medical system 10 comprising an implantable medical device (IMD) 12, and implantable medical leads 14, 16 electrically coupled to IMD 12. In the embodiment shown in FIG. 1, system 10 is implanted within a patient 18 to deliver electrical stimulation therapy to the heart 5 of patient 18. Patient 18 ordinarily, but not necessarily, will be a human patient.

In the embodiment shown in FIG. 1, IMD 12 is a pacemaker. Leads 14, 16 each include at least one electrode positioned within, e.g., intravenously, or proximate to, e.g., epicardially, heart 5 in order to deliver therapeutic electrical stimulation from IMD 12 to heart 5. The therapeutic electrical stimulation may include, for example, pacing or defibrillation pulses, or continuous time signals. In some embodiments, at least one of leads 14, 16 may provide stimulation to heart 5 without contacting heart 5. For example, one or more of leads 14, 16 may be implanted subcutaneously, outside of the chest cavity, and deliver stimulation to heart 5. Although illustrated in FIG. 1 as including two leads 14, 16, systems according to the present disclosure may include any number of leads coupled to a medical device.

In various embodiments, IMD 12 may comprise any of a wide variety of medical devices that are configured to couple to one or more medical leads and deliver electrical stimulation therapy to patient 18 via the leads. As non-limiting examples, IMD 12 may be an implantable cardiac pacemaker that provides therapeutic stimulation to heart 5, an implantable cardioverter, an implantable defibrillator or an implantable cardiac pacemaker-cardioverter-defibrillator (PCD). IMD 12 may deliver pacing, cardioversion or defibrillation signals to patient 18 via electrodes disposed proximate to the distal ends of one or more leads 14, 16. Accordingly, in different embodiments, leads 14, 16 may electrically couple one or more electrodes to IMD 12, and leads 14, 16 may be positioned to deliver therapeutic electrical signals (e.g., pulses or continuous signals) to various cardiac locations.

In the example illustrated by FIG. 1, lead 14 extends from IMD 12 into the right ventricle (RV) of heart 5, and lead 16 extends from IMD 12 into the coronary sinus and sub-branches proximate to the left ventricle (LV) of heart 5. IMD 12 may provide bi-ventricular pacing and, in some embodiments, cardiac resynchronization therapy (CRT) via leads 14, 16. As described in greater detail below, one or both of leads 14, 16 may include features, such as at least one recessed electrode, that facilitate control of the depth of propagation of a stimulation field from the leads. One or more recessed electrodes may be particularly useful in the case of an LV lead 16, where the electrodes may be located proximate to the phrenic nerve of patient 18, because a stimulation field with a limited depth of propagation may avoid capture of the phrenic nerve.

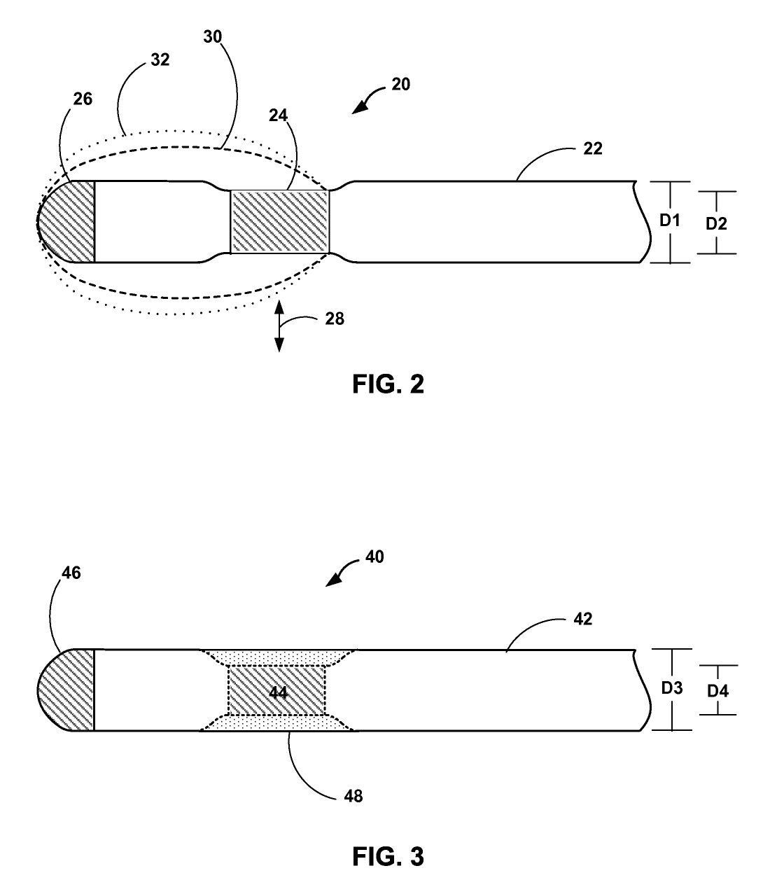

FIG. 2 is a side view of an embodiment of a distal end of a lead 20, which may, for example, correspond to either of leads 14, 16 of FIG. 1. Lead 20 includes electrodes 24 and 26. Lead 20 also includes a lead body 22 including one or more elongated conductors (not shown) covered or surrounded by one or more elongated insulative bodies. The electrodes 24, 26 are coupled to the conductors and are not covered by the insulative body or covering. Allowing electrodes 24, 26 to be exposed to tissue of the patient allows data to be sensed from the tissue and/or therapy delivered to the patient. A proximal end (not shown) of lead 20 may be coupled to an IMD (e.g., IMD 12 of FIG. 1). In some embodiments, electrodes 24 and 26 may be ring electrodes, each with a substantially circular cross-section. In other embodiments, electrodes 24 and 26 may comprise segmented or partial ring electrodes, each of the electrode segments extending along an arc less than 360 degrees (e.g., 90-120 degrees). Segmented or partial ring electrodes may be useful for providing an electrical stimulation field in a particular direction and/or targeting a particular stimulation site. The configuration, type, and number of electrodes 24 and 26 illustrated in FIG. 2 are merely exemplary.

In the embodiment illustrated in FIG. 2, electrode 24 is arranged as a ring, and more particularly is a ring electrode in the illustrated example. In the embodiment illustrated in FIG. 2, electrode 24 is recessed relative to lead body 22. More particularly, the diameter D2 of electrode 24, i.e., the diameter of the ring, is smaller than the diameter D1 of lead body 22 such that electrode 24 is recessed relative to lead body 22. For example, diameter D2 of recessed electrode 24 may be about 0.01 mm to about 0.5 mm less than the diameter D1 of lead body 22. As another example, the ratio of diameter D1 of lead body 22 to D2 of recessed electrode 24 may range from about 10 to 9 to about 10 to 8. Recessed electrode 24 may aid in limiting the distance a stimulation field propagates or extends from an outer diameter of lead body 22, in radial direction 28 perpendicular to the longitudinal axis of lead body 22, relative to an electrode having a diameter D1 equal to diameter D2 of lead body 22. The distance a stimulation field extends from an outer diameter of lead body 22 in radial direction 28 perpendicular to the longitudinal axis of lead body 22 may also be referred to as the depth of propagation of the stimulation field. As one example, when electrode 26 is configured as a cathode and recessed electrode 24 is configured as an anode, outline 30 may represent the outer boundaries of the stimulation field. In contrast, using the same anode and cathode configuration but extending electrode 24 radially outward in direction 28 until the diameter D2 of electrode 24 equals the diameter D1 of lead body 22 may yield a stimulation field with outer boundaries 32. In this manner, recessed electrode 24 provides a stimulation field having a limited depth extending from an outer diameter of lead body 22. The recessed electrode 24 draws the stimulation field closer to the longitudinal axis of lead body 22. In this manner, the relationship between diameter D2 of electrode 24 and D1 of lead body 22 may aid in controlling the depth of propagation of the stimulation field.

A stimulation field with limited depth may be useful in preventing unintended and undesirable stimulation of nerves and/or muscles outside the proximity of lead body 22. As one example, a field with a limited depth may be particularly useful in left ventricle (LV) pacing applications. During LV pacing applications, lead 20, and more specifically electrodes 24 and 26 of lead 20, may be positioned proximate to the phrenic nerve. Using recessed electrode 24 to limit the depth of the stimulation field may help prevent stimulation of the phrenic nerve, while still enabling capture of LV myocardial tissue by pacing stimulation.

At least a portion of lead 20, such as electrodes 24, 26 or a separate marker loaded in or formed on lead body 22, may include a radio-opaque material that is detectable by imaging techniques, such as fluoroscopic imaging or x-ray imaging. For example, electrodes 24, 26 may be made of platinum iridium, which is detectable via imaging techniques. This feature may be helpful for maneuvering lead 20 relative to a target site within the body. Radio-opaque markers, as well as other types of markers, such as other types of radiographic and/or visible markers, may also be employed to assist a clinician during the introduction and withdrawal of stimulation lead 40 from a patient. Markers identifying the location of each electrode may be particularly helpful. Since the electrodes rotate with the lead body, a clinician may rotate the lead and the electric field to stimulate a desired tissue. Markers may help guide the rotation.

FIG. 3 is a side view of another embodiment of a distal end of a lead 40. Lead 40 is similar to lead 20 of FIG. 2 but, as described in further detail below, includes a shield 48 over the recessed electrode 44. Lead 40 includes a lead body 42 with an outer diameter D3. A proximal end (not shown) of lead 40 may be coupled to an IMD (e.g., IMD 12 of FIG. 1). Lead 40 also includes electrodes 44 and 46. In some embodiments, electrodes 44 and 46 may be ring electrodes, each with a substantially circular cross-section. In other embodiments, electrodes 44 and 46 may comprise segmented or partial ring electrodes. Additionally, lead 40 may include any configuration, type, and number of electrodes 44 and 46 and is not limited to the embodiment illustrated in FIG. 3.

In the embodiment illustrated in FIG. 3, electrode 44 is recessed relative to lead body 42. More particularly, the diameter D4 of electrode 44 is smaller than the diameter D3 of lead body 42 such that electrode 44 is recessed relative to lead body 42. For example, diameter D4 of recessed electrode 44 may be about 0.01 mm to about 0.5 mm less than the diameter D3 of lead body 42. As another example, the ratio of diameter D3 of lead body 42 to D4 of recessed electrode 44 may range from about 10 to 8 to about 10 to 9. As another example, the proportionality of diameters or D3/D4 of lead body 42 can range from about 20 to 1.26. Like recessed electrode 24, recessed electrode 44 may provide a stimulation field having a limited depth extending from an outer diameter of lead body 42. Shield 48 may be positioned on an outer surface of recessed ring electrode 44 such that an outer diameter of shield 48 is substantially flush or flush with lead body 42. This allows lead 40 to be isodiametric throughout the length of lead body 42, which may be helpful in preventing thrombosis. Allowing lead 40 to be isodiametric throughout the length of lead body 42 may also make implantation of lead 40 easier. In some embodiments, shield 48 may be made of a polymer such as, for example, expanded PTFE, urethane, and silicone. Also, in some embodiments, shield 48 may include perforations that allow an electrical stimulation signal to propagate between electrode 44 and patient tissue proximate to lead 40.

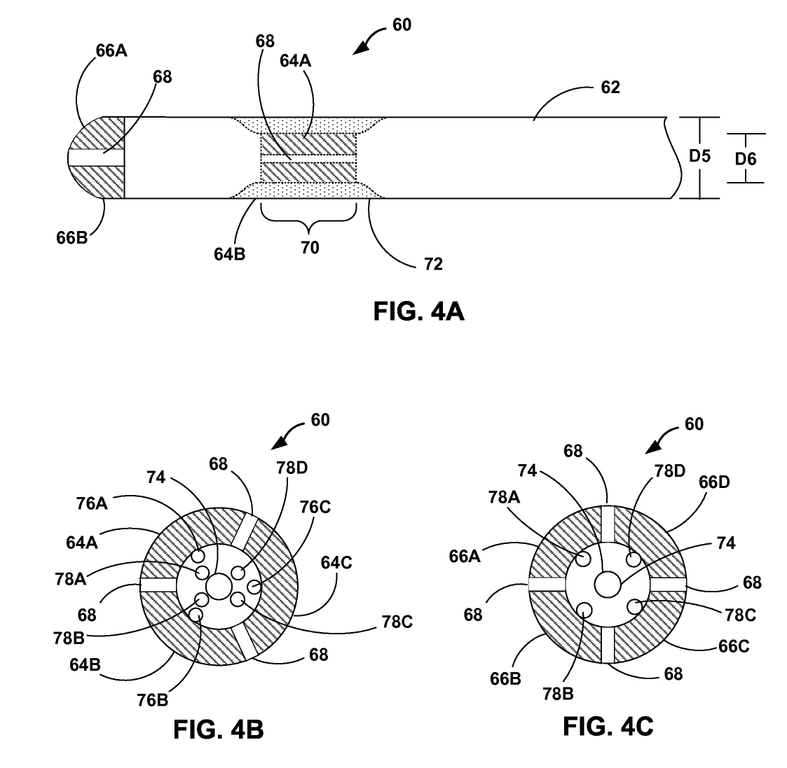

FIGS. 4A-4C are a side view, and two cross-sectional views of an embodiment of a distal end of a lead 60. Lead 60 is similar to lead 30 of FIG. 3 but, as described in further detail below, includes electrode segments 64A-64C arranged as a recessed ring and electrodes segments 66A-66D at its distal tip (64C, 66C and 66D not shown). Lead 60 includes a lead body 62 with an outer diameter D5. A proximal end (not shown) of lead 60 may be coupled to an IMD (e.g., IMD 12 of FIG. 1). The electrode segments may be separated from one another by an insulative, nonconductive material 68. Insulative material 68 is a biocompatible material having an impedance sufficient to prevent shorting between electrode segments during stimulation therapy. For example, insulative material 68 may comprise polyurethane, silicone, and fluoropolymers such as tetrafluroethylene (ETFE), polytetrafluroethylene (PTFE), and/or expanded PTFE (i.e. porous ePTFE, nonporous ePTFE).

Electrode segments 64A-64C are positioned to form a ring 70 with a diameter D6 and a substantially circular cross-section. For example, electrodes 64A-64C are located at substantially the same axial position along the length of lead body 62, but each of electrodes 64A-64C has a different radial position. However, rings according to the present disclosure are not limited to configurations with electrodes at substantially the same axial position. According to one embodiment of the disclosure, electrode segments of a ring may be staggered or otherwise positioned at multiple axial positions of the lead body.

In the embodiment illustrated in FIGS. 4A-4C, diameter D6 of ring 70 is smaller than the diameter D5 of lead body 62 such that electrode segments 64A-64C are each recessed relative to lead body 62. For example, diameter D6 of recessed ring 70 may be about 0.01 mm to about 0.5 mm less than the diameter D5 of lead body 62. As another example, the ratio of diameter D5 of lead body 62 to D6 of recessed ring 70 may range from about 10 to 8 to about 10 to 9. One or more of electrode segments 64A-64C of recessed ring 70 may be activated to provide a stimulation field having a limited depth extending from an outer diameter of lead body 62. Electrodes 64A-64C are shown as an example of configuration of ring 70. However, in other embodiments, ring 70 may include more or less than three electrode segments. In general, in embodiments in which a ring includes electrode segments, at least two electrode segments may be positioned to form the ring, e.g., ring 70.

Shield 72 is positioned on an outer surface of recessed ring 70, and more specifically over electrode segments 64A-64C, such that an outer diameter of shield 72 is substantially flush with lead body 62. Shield 72 may allow lead 60 to be isodiametric along the length of lead body 72, which may be helpful in preventing thrombosis. In some embodiments, shield 72 may include perforations that allow an electrical stimulation signal to transfer from electrode segments 64A-64C to a target stimulation site proximate to lead 60.

In the illustrated embodiment, lead 60 also includes electrode segments 66A-66D distal to recessed ring 70. Using a bipolar configuration, one or more of electrode segments 66A-66D and one or more of electrode segments 64A-64C may be activated to create an electrical stimulation field. In other embodiments, lead 60 may include additional electrodes (e.g., partial ring electrodes, electrode segments positioned to form a ring, or ring electrodes) at various axial positions along the length of lead body 62. In yet other embodiments, lead 60 may include fewer electrodes. For example, lead 60 may only include electrode segments 64A-64C positioned on ring 70. In some embodiments, the IMD (e.g., IMD 12 of FIG. 1) or a conductive portion (e.g., a coil electrode) on another lead (e.g., a lead with a distal end implanted in the right ventricle) may be activated as a cathode or an anode in a unipolar configuration. As one example, a superior vena cava (SVC) coil and/or a right ventricle (RV) coil of a lead with a distal end implanted in the right ventricle may be activated as an anode or a cathode.

FIG. 4B is a cross-sectional view of lead 60 showing ring 70 including electrode segments 64A-64C. As previously described, each of electrode segments 64A-64C is separated by insulative material 68. The center of lead body 62 may include a lumen 74 to accommodate a delivery device such as a stylet, guidewire or a hybrid of a stylet and guidewire. A delivery device may be used to help position lead 60 at a target location during implantation of lead 60. Lead 60 also includes electrical conductors 76A-76C coupled to electrode segments 64A-64C respectively and electrical conductors 78A-78D coupled to electrode segments 66A-66D respectively. Each of conductors 76A-76C extends from ring 70 to a proximal end of lead 60 to couple electrodes 64A-64C to an IMD (e.g., IMD 12 of FIG. 1). Likewise, each of conductors 78A-78D extends from the distal tip of lead 60 where electrodes 66A-66D are positioned to a proximal end of lead 60 to couple electrodes 66A-66D to an IMD (e.g., IMD 12 of FIG. 1).

FIG. 4C is a cross-sectional view of the distal tip of lead 60 including electrode segments 66A-66D. FIG. 4C also illustrates conductors 78A-78D coupled to electrode segments 66A-66D, respectively, and lumen 74 within lead body 62.

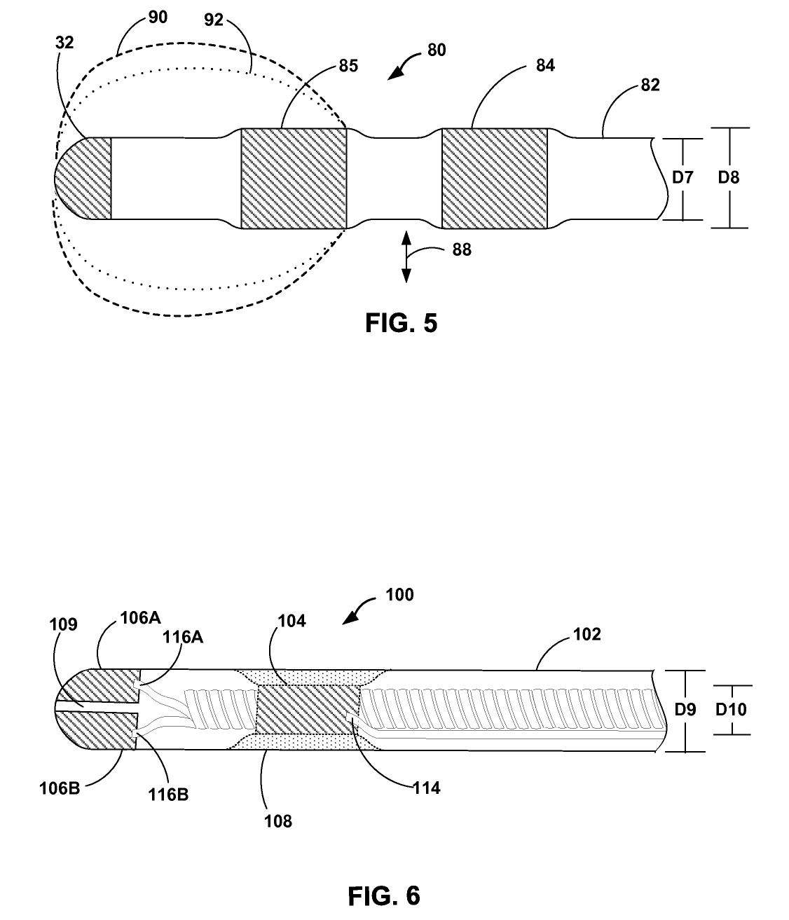

FIG. 5 is a side view of an embodiment of a distal end of a lead 80. Lead 80 includes a lead body 82 with an outer diameter D7. A proximal end (not shown) of lead 70 may be coupled to an IMD (e.g., IMD 12 of FIG. 1). Lead 80 also includes electrodes 84, 85 and 86. In some embodiments, electrodes 84, 85 and 86 may be ring electrodes, each with a substantially circular cross-section. In other embodiments, electrodes 84, 85 and 86 may comprise segmented or partial ring electrodes arranged as a ring, each of which extends along an arc less than 360 degrees (e.g., 90-120 degrees). Segmented or partial ring electrodes may be useful for providing an electrical stimulation field in a particular direction and/or targeting a particular stimulation site. Additionally, lead 80 may include any configuration, type, and number of electrodes 84, 85 and 86 and is not limited to the embodiment illustrated in FIG. 5.

In the embodiment illustrated in FIG. 5, electrodes 84 and 85 protrude relative to lead body 82. More particularly, the diameter D8 of electrodes 84 and 85 is larger than the diameter D7 of lead body 82 such that electrodes 84 and 85 protrude relative to lead body 82. For example, diameter D8 of electrodes 84 and 84 may be about 0.01 mm to about 0.5 mm greater than the diameter D7 of lead body 82. As another example, the ratio of diameter D7 of lead body 82 to D8 of protruded electrodes 84 and 85 may range from about 8 to 10 to about 9 to 10. Protruded electrodes 84 and 85 may aid in increasing the distance a stimulation field extends from an outer diameter of lead body 82 in radial direction 88, which is perpendicular to the longitudinal axis of lead body 82 relative to an electrode having a diameter D8 equal to diameter D7 of lead body 82. The distance a stimulation field extends from an outer diameter of lead body 82 in radial direction 88 perpendicular to the longitudinal axis of lead body 82 may also be referred to as the depth of the stimulation field. As one example, when electrode 86 is configured as a cathode and protruded electrode 85 is configured as an anode, outline 90 may represent the outer boundaries of the stimulation field. In contrast, using the same anode and cathode configuration but moving electrode 95 radially inward in direction 88 until the diameter D8 of electrode 85 equals the diameter D7 of lead body 82 would yield a stimulation field with outer boundaries 92. The protruded electrode 85 extends the stimulation field farther from the longitudinal axis of lead body 82. In this manner, the relationship between diameter D8 of electrode 85 and D7 of lead body 82 may aid in controlling the depth of the stimulation field. A stimulation field with increased depth may be useful in delivering stimulation to a target stimulation site further from lead body 82 than reachable if the diameter D8 of electrode 85 equaled the diameter D7 of lead body 82.

FIG. 6 is a side view of an embodiment of a distal end of a lead 100. Lead 100 includes a lead body 102 with an outer diameter D9. A proximal end (not shown) of lead 100 may be coupled to an IMD (e.g., IMD 12 of FIG. 1). Lead 100 also includes electrodes 104, 106A, and 106B. In the embodiment illustrated in FIG. 6, electrode 104 is a ring electrode with a substantially circular cross-section, and electrodes 106A and 106B are segmented or partial ring electrodes, each extending along an arc less than 360 degrees (e.g., 90-120 degrees). Electrodes 106A and 106B may be electrically insulated from each other by insulative material 109. For example, insulative material 109 may comprise polyurethane, silicone, and fluoropolymers such as tetrafluroethylene (ETFE), polytetrafluroethylene (PTFE), and/or expanded PTFE (i.e. porous ePTFE, nonporous ePTFE). Additionally, electrode 104 is recessed relative to lead body 102 (i.e., diameter D9 of electrode 104 is less than diameter D10 of lead body 102) and is covered by shield 108, which may be substantially similar to shields 48 and 72 of FIGS. 3 and 4A, respectively. Recessed electrode 104 may be activated to provide a stimulation field having a limited depth extending from an outer diameter of lead body 102. In other embodiments, lead 100 may include any configuration, type, and number of electrodes 104, 106A and 106B and is not limited to the embodiment illustrated in FIG. 6. For example, lead 100 or any other lead according to the disclosure may include any number of recessed or protruded rings, each ring including at least one electrode. As one example, lead 100 may include two or more recessed rings.

Lead 100 also includes electrical conductors 114, 116A, and 116B electrically coupled to electrodes 104, 106A, and 106B, respectively. In the illustrated embodiment, conductors 116A and 116B are coiled along the length of lead body 102, and conductor 114 lays axial to conductors 116A and 116B. In the embodiment illustrated in FIG. 6, each of conductors 114, 116A, and 116B is electrically coupled to a single one of electrodes 104, 106A and 106B, respectively. In this manner, each of electrodes 104, 106A, and 106B may be independently activated.

The configuration, type, and number of conductors 114, 116A, and 116B is not limited to the embodiment illustrated in FIG. 6 and, in other embodiments, lead 100 may include any configuration, type, and number of conductors. As one example, in some embodiments, each of conductors 114, 116A, and 116B may be coiled conductors. Additionally or alternatively, one conductor may be electrically coupled to two or more electrodes. Additionally, each of leads 12, 14, 20, 40, 60, and 80 may include conductors to electrically couple its electrodes at the distal end of its lead body to an IMD (e.g., IMD 12 of FIG. 1) couple to the proximal end of its lead body.

Various examples have been described. However, modification may be made to the described examples. For example, leads used in conjunction with the techniques described herein may include fixation mechanisms, such as tines that passively secure a lead in an implanted position or a helix located at a distal end of the lead that required rotation of the lead during implantation to secure the helix to a body tissue. Further, although depicted herein as being located at a distal end of a lead body, in other examples a ring with one or more electrodes an outer diameter different than an outer diameter of a lead body be located at any axial position of the lead body. These and other examples are within the scope of the following claims.

1. An implantable electrical stimulation lead comprising:
a lead body; and
at least one electrode arranged as a ring, wherein an outer diameter of the ring being recessed from an outer diameter of the lead body.

2. The lead of claim 1, wherein the at least one electrode comprises a single ring electrode.

3. The lead of claim 1, wherein the at least one electrode comprises at least two electrode segments.

4. The lead of claim 1, wherein the outer diameter of the ring being about 0.01 mm to about 0.5 mm smaller than the outer diameter of the lead body.

5. The lead of claim 1, wherein a ratio of the outer diameter of the lead body to the outer diameter of the ring being about 10 to 8.

6. The lead of claim 1, further comprising a shield positioned on an outer surface of the ring over the at least one electrode, wherein an outer diameter of the shield being about flush with the lead body.

7. The lead of claim 6, wherein the shield comprises at least one perforation that allows a stimulation signal to propagate between the at least one electrode and tissue.

8. The lead of claim 1, wherein a ratio of the outer diameter of the lead body to the outer diameter of the ring being within a range of between 20:1 to 10:9.

9. The lead of claim 1, wherein the least one electrode comprises at least one first electrode, further comprising a second electrode axially displaced from the ring along a length of the lead body.

10. The lead of claim 9, wherein the second electrode being positioned distal to the ring.

11. An implantable electrical stimulation lead comprising:
a lead body; and
at least one electrode arranged as a ring, wherein an outer diameter of the ring being greater than the lead diameter such that the ring protrudes relative to the lead body.

12. The lead of claim 11, wherein the outer diameter of the ring being about 0.01 mm to about 0.5 mm greater than the outer diameter of the lead body.

13. The lead of claim 11, wherein a ratio of the outer diameter of the ring to the outer diameter of the lead body being within a range of between 20:1 to 10:9.

14. A system comprising:
a medical device that delivers electrical stimulation; and
an implantable electrical stimulation lead, wherein the lead comprises:
a lead body; and
at least one electrode arranged as a ring, wherein an outer diameter of the ring differs from an outer diameter of the lead body.

15. The system of claim 14, wherein the at least one electrode comprises a single ring electrode.

16. The system of claim 14, wherein the at least one electrode comprises at least two electrode segments.

17. The system of claim 14, wherein the outer diameter of the ring being smaller than the outer diameter of the lead body such that the at least one electrode being recessed relative to the lead body.

18. The system of claim 17, wherein the outer diameter of the ring being about 0.01 mm to about 0.5 mm smaller than the outer diameter of the lead body.

19. The system of claim 17, wherein a ratio of the outer diameter of the lead body to the outer diameter of the ring being about 10 to 8.

20. The system of claim 17, further comprising a shield positioned on an outer surface of the ring over the at least one electrode, wherein an outer diameter of the shield being substantially flush with the lead body.

21. The system of claim 20, wherein the shield comprises at least one perforation that allows a stimulation signal to propagate between the at least one electrode and tissue within which the lead is implanted.

22. The system of claim 14, wherein the ring diameter is greater than the lead diameter such that the ring protrudes relative to the lead body.

23. The system of claim 22, wherein the outer diameter of the ring is about 0.01 mm to about 0.5 mm greater than the outer diameter of the lead body.

24. The system of claim 22, wherein a ratio of the outer diameter of the lead body to the outer diameter of the ring is about 8 to 10.

25. The system of claim 14, wherein the least one electrode comprises at least one first electrode, further comprising a second electrode axially displaced from the ring along a length of the lead body.

26. The system of claim 25, wherein the second electrode is positioned distal to the ring.

27. The system of claim 14, wherein the medical device comprises a cardiac stimulator.

28. The system of claim 14, wherein the medical device comprises an implantable medical device.

29. A method comprising:
implanting an electrical stimulation lead within a patient, the lead comprising:
a lead body, and
at least one electrode arranged as a ring, wherein an outer diameter of the ring being recessed from an outer diameter of the lead body; and
delivering stimulation therapy to a tissue within the patient using the at least one electrode.

30. An implantable electrical stimulation lead comprising:
a lead body; and
means for providing a stimulation field having a limited depth extending from an outer diameter of the lead body.
